(19) United States
(12) Patent Application Publication
(10) Pub. No.: US 2021/0073417 A1
(43) Pub. Date: Mar. 11, 2021

(54) Case storage system

(71) Applicant: HEART ORGANIZATION CO., LTD., Osaka (JP)

(72) Inventors: Toshiko SUGAWARA, Osaka (JP); Tetsuya KANEUCHI, Osaka (JP)

(21) Appl. No.: 16/743,277

(22) Filed: Jan. 15, 2020

(30) Foreign Application Priority Data
Sep. 10, 2019 (JP) 2019-164082

(51) Int. Cl. G06F 21/62; G16H 10/60

(52) U.S. Cl. CPC G06F 21/6254; G16H 10/60

(57) ABSTRACT

To provide a case storage device capable of transmitting/storing case data to/in a server device from any type of a terminal device without a risk of leakage of a privacy item. When receiving an upload-related page request, upload preparation means 6 transmits a mask processing program 24 to a terminal device T. Mask processing program recording means 18 in the terminal device T temporarily stores the received mask processing program 24 in a recording section 22. Case data transmission means 20 masks privacy information in case data recorded in the recording section 22 by the mask processing program 24, and transmits the case data to a server device S. Thus, the masked case data is transmitted to the server device S even when an operator on the terminal device T side is unconscious of masking. Recording means 8 in the server device S records the received masked case data in a recording section 2. In this way, even when a special program is not prepared on the terminal device T side in advance, it is possible to communicate and store the case data containing a masked privacy item.

### BACKGROUND OF THE INVENTION

### 1. Field of the Invention

The present invention relates to a system for storing case data.

### 2. Related Art

In the case where case data is shared among many persons, for example, in the case where the case data is stored in a server device on the Internet for a research purpose and shared, not only data required for case research but also information on personal privacy are transmitted and stored.

In order to prevent this, for example, in JP-A-2005-184731, the case data is transmitted in the server device or the like after the information on the privacy is deleted at the time of the transmission. In this way, the case data can be transmitted and stored while the privacy is protected.

### SUMMARY OF THE INVENTION

However, in the related art as disclosed in Patent Document 1, it is required to prepare in advance a special function to delete a privacy item for a terminal device on the transmission side. Thus, there is a problem that the safe transmission cannot be achieved by the terminal device without such a function.

In the case where the terminal device that cannot delete the privacy item is used, the privacy item has to be deleted by the server device or the like on a reception side. While such a method has an advantage that the case data can be transmitted from any type of the terminal device, there is a problem that the privacy item is contained in the transmitted data, which develops a risk.

The present invention solves a problem as described above and therefore has a purpose of providing a system capable of transmitting/storing case data to/in a server device from any type of a terminal device without a risk of leakage of a privacy item.

Characteristics of the present invention that can independently be applied will be listed below.

(1) to (5) A case storage system according to the present invention is a case storage system including a case server device and a terminal device.

The case server device includes: upload preparation means that receives a request for an upload command related page from the terminal device and transmits said upload command related page and a mask processing program to said terminal device; case transmission means that receives a case request from the terminal device and transmits masked case data recorded in a recording section to said terminal device; and recording means that records the transmitted masked case data in the recording section.

The terminal device includes: upload command related page request means that transmits the upload command related page request to the case server device; display means that receives and displays the upload command related page request, which is transmitted from the case server device in response to the upload command related page request, on a display section, and displays the masked case data, which is transmitted from the case server device in response to the case request, on the display section; mask processing program recording means that receives the mask processing program, which is transmitted from the case server device in response to the upload command related page request, and records the mask processing program at least temporarily in the recording section; and case data transmission means that transmits the masked case data, in which specified privacy information in the case data is masked by the mask processing program, to the case server device when receiving upload command input for uploading the case data to the case server device on the basis of the upload command related page.

Thus, even when special software is not prepared on the terminal device side in advance, it is possible to mask the privacy information, and upload/store the case data to/in the case server device.

(6) In the case storage system according to the present invention,
instead of receiving the upload command related page request, the upload preparation means acquires that the upload command is entered in the terminal device, and transmits the mask processing program.

Thus, the mask processing program is not transmitted until uploading is actually commanded. Therefore, it is possible to eliminate wasteful transmission processing.

(7) In the case storage system according to the present invention, masking is performed by deleting an item itself, deleting a content of the data, or replacing the content of the data with a masking character.

Thus, it is possible to upload by preventing leakage of a privacy item.

(8) In the case storage system according to the present invention, masking is performed by at least masking the item such as a patient's name, birth date, medical facility information, or primary doctor information among the items of the case data.

Thus, it is possible to prevent leakage of the patient's name.

(9) In the case storage system according to the present invention, the case data is DICOM data.

Thus, it is possible to upload the DICOM data while preventing the leakage of the privacy item.

(10) (11) The case storage server according to the present invention includes: upload preparation means that receives a request for an upload command related page from a terminal device and transmits said upload command related page and a mask processing program to said terminal device; and recording means that records the transmitted masked case data in a recording section.

Thus, even when the special software is not prepared on the terminal device side in advance, it is possible to mask the privacy information, and upload/store the case data to/in the case server device.

In an embodiment, steps S60, S61 correspond to the “upload preparation means”.

In the embodiment, step S53 corresponds to the “case transmission means”.

In the embodiment, step S62 corresponds to the “recording means”.

In the embodiment, step S10 corresponds to the “upload command related page request means”.

In the embodiment, step S6 corresponds to the “display means”.

In the embodiment, step S12 corresponds to the “mask processing program recording means”.

In the embodiment, step S16 corresponds to the “case data transmission means”.

The “upload command related page” is a concept that includes not only a page provided with an upload command button but also a page having a direct or indirect link to the upload command button among pages recorded in the case server device.

The “program” is a concept that includes not only a program that can directly be executed by a CPU but also a program in a source format, a program subjected to compression processing, an encrypted program, and the like.

The features of the present invention can be described broadly as set forth above. The structures and characteristics of the present invention will be apparent from the following detailed description of the present invention together with those features, effects, and drawings.

### BRIEF DESCRIPTION OF THE DRAWINGS

FIG. 1 is a functional configuration diagram of a case storage system according to an aspect of the present invention.

FIG. 2 is a system configuration diagram of the case storage system.

FIG. 3 illustrates a hardware configuration of a case server device.

FIG. 4 illustrates a hardware configuration of a terminal device.

FIG. 5 illustrates a flowchart of case browsing processing.

FIGS. 6A-6C illustrate examples of a display screen on the terminal device in the case browsing processing.

FIG. 7A illustrates a data example of a case DB 44.

FIG. 7B illustrates a data example of the case DB 44.

FIG. 8 is a flowchart of case registration processing.

FIG. 9A illustrates an example of DICOM data.

FIG. 9B is an example of the masked DICOM data.

FIG. 10 is an example of a case registration screen.

### DESCRIPTION OF EMBODIMENT

### 1. Functional Configuration

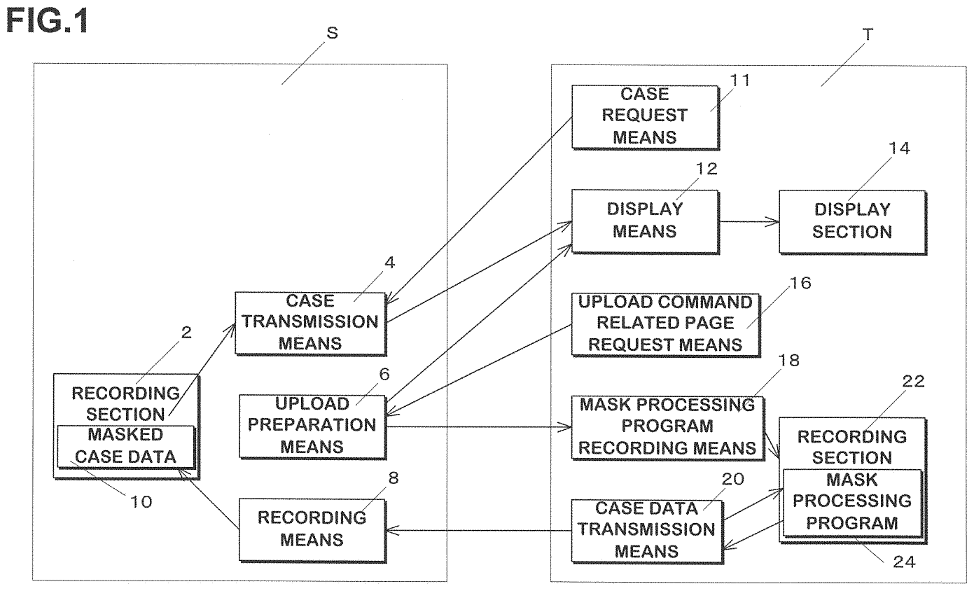

FIG. 1 is a functional configuration diagram of a case storage system according to an aspect of the present invention. Processing is executed as follows when a case is browsed from a terminal device T. Case request means 11 in the terminal device T transmits a case request to a server device S. Case transmission means 4 in the server device S receives the case request and transmits masked case data 10 recorded in a recording section 2. Display means 12 in the terminal device T displays the received masked case data 10 on a display section 14. In this way, of the cases stored in the server device S, the requested case can be displayed on the terminal device T.

Processing is executed as follows when case data is uploaded from the terminal device T. Upload command related page request means 16 in the terminal device T transmits a request for an upload-related page to the server device S. Upload preparation means 6 in the server device S receives this request and transmits the upload-related page to the terminal device T. The display means 12 in the terminal device T displays the received upload-related page on the display section 14.

When receiving the request for the upload-related page, the upload preparation means 6 transmits a mask processing program 24 to the terminal device T. Mask processing program recording means 18 in the terminal device T temporarily records the received mask processing program 24 in a recording section 22.

Case data transmission means 20 masks privacy information in case data, which is recorded in the recording section 22, using the above mask processing program 24, and transmits the case data to the server device S. Thus, the masked case data is transmitted to the server device S even when an operator on the terminal device T side is unconscious of masking.

Recording means S in the server device S records the received masked case data in the recording section 2. In this way, even when a special program is not prepared on the terminal device T side in advance, it is possible to communicate and store the case data containing a masked privacy item.

### 2. System Configuration

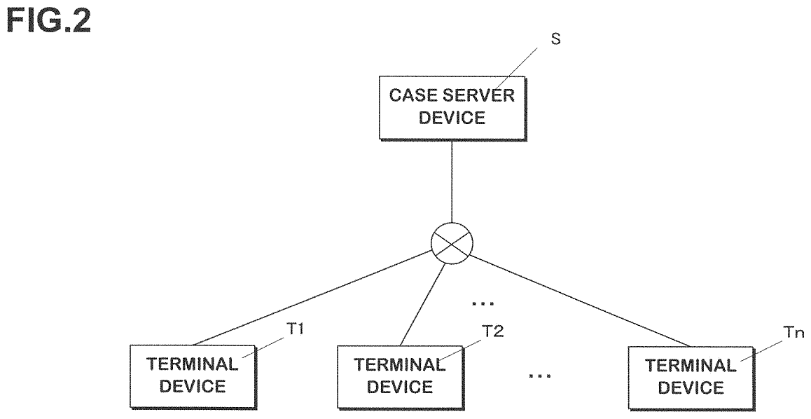

FIG. 2 illustrates a system configuration of the case storage system. Terminal devices T1, T2 . . . Tn are connected to the case server device S via the Internet. For example, the terminal devices T1, T2 . . . Tn are PCs used by doctors at many different hospitals and the like. The cases stored in the case server device S can be browsed from each of the terminal devices T1, T2 . . . Tn.

In addition, DICOM data can be uploaded as the case data to the server device S from each of the terminal devices T1, T2 . . . Tn.

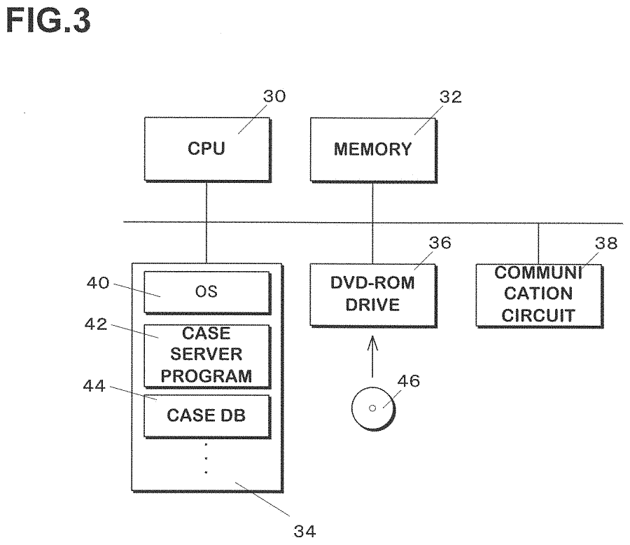

FIG. 3 illustrates a hardware configuration of the case server device S. Memory 32, a hard disk 34, a DVD-RCM drive 36, and a communication circuit 38 are connected to a CPU 30. The communication circuit 38 is used to connect to the Internet.

In the hard disk 34, an operating system 40, a case server program 42, and a case DB 44 are recorded. The case server program 42 fulfills its function in cooperation with the operating system 40. These programs are recorded in a DVD-ROM 46 and are installed in the hard disk 34 via the DVD-ROM drive 36.

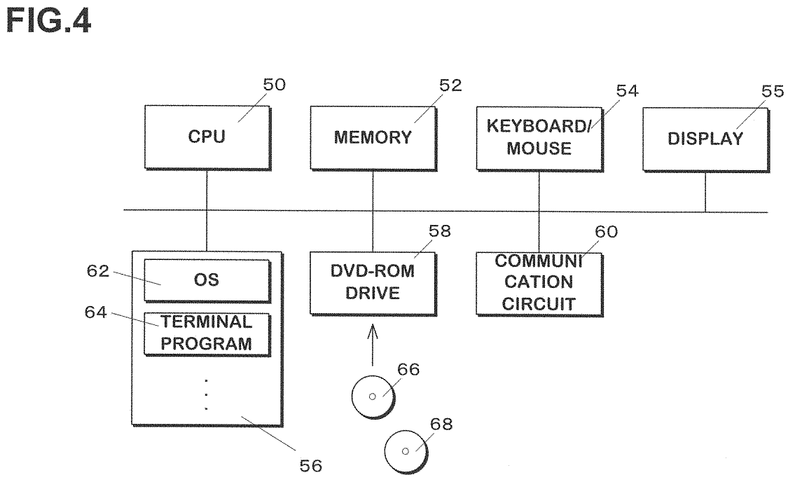

FIG. 4 illustrates a hardware configuration of the terminal device T. Memory 52, a keyboard/mouse 54, a display 55, a hard disk 56, a DVD-ROM drive 58, and a communication circuit 60 are connected to a CPU 50. The memory 52 is a temporary storage section that functions as a work area of the CPU 50. The communication circuit 60 is used to connect to the Internet.

In the hard disk 56, an operating system 62 and a terminal program 64 are recorded. The terminal program 64 fulfills its function in cooperation with the operating system 62. These programs are recorded in a DVD-ROM 66 and are installed in the hard disk 56 via the DVD-ROM drive 58.

### 3. Case Browsing Processing

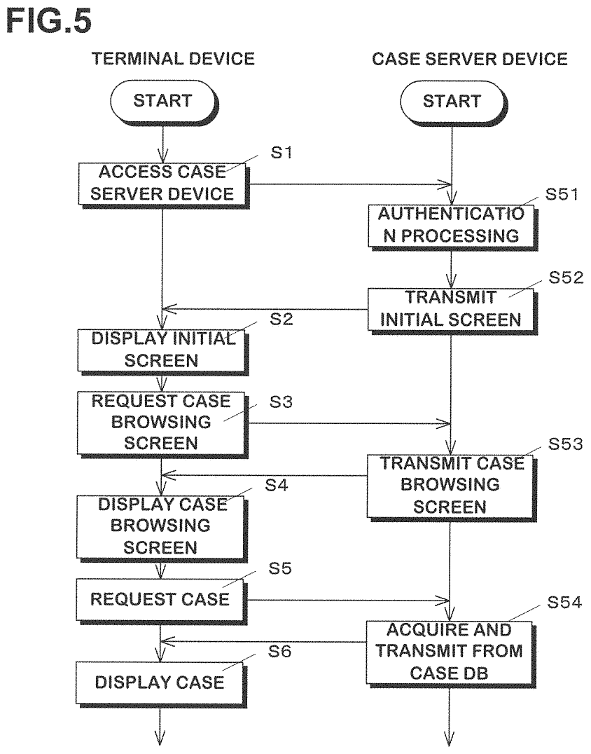

FIG. 5 illustrates a flowchart of case browsing processing. A user (the doctor or the like) who operates the terminal device T operates the keyboard/mouse 54 of the terminal device T to input a command to access the case server device S. At this time, a predetermined user ID and a predetermined password are also input.

In response to this, the CPU 50 in the terminal device T (hereafter may simply be described as the terminal device T) accesses the case server device S (step S1).

The case server device S performs personal authentication on the basis of the transmitted user ID and the transmitted password (step S51). If a result of the authentication is not OK, later processing is not executed, and the case browsing processing is terminated.

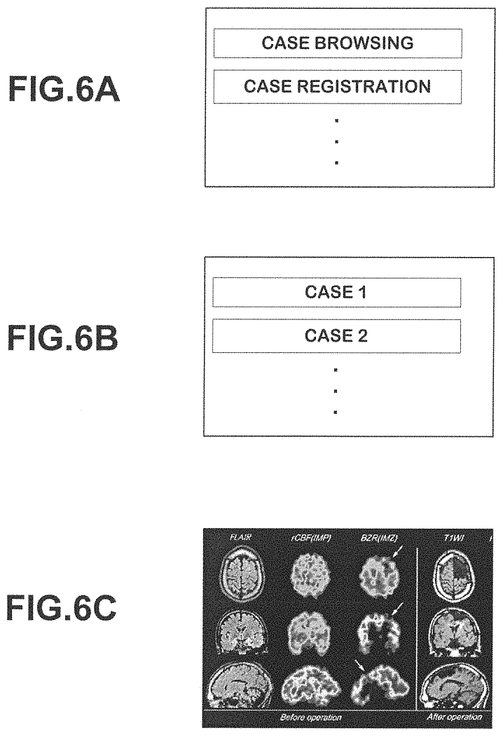

If the result of the authentication is OK, the CPU 30 in the case server device S transmits an initial screen to the terminal device T (step S52). The terminal device T displays this initial screen on the display 55 (step S2). An example of the initial screen is illustrated in FIG. 6A. Menus such as case browsing and case registration are displayed.

When the operator of the terminal device T selects the case browsing, the terminal device T transmits a request for a case browsing screen to the server device S (step S3). In response to this, the case server device S extracts a disease name and the like from the case DB 44 recorded in the hard disk 34, generates and transmits a case browsing screen (step S53). Note that, instead of the hard disk 34, non-volatile memory (such as an SSD) or the like may be used as an auxiliary storage device.

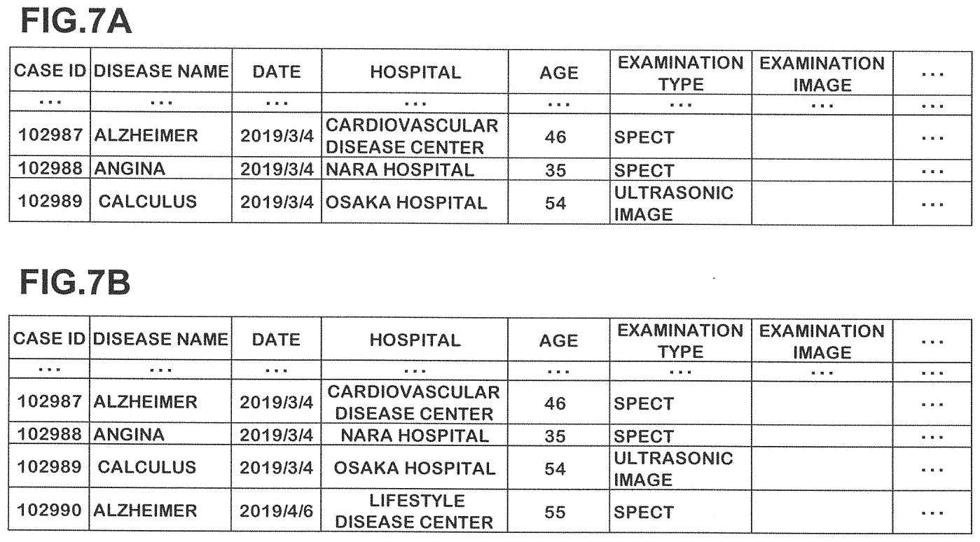

FIG. 7A illustrates an example of the case DB 44. The disease name, a date, the hospital, age, an examination type, an examination image, and the like are recorded in association with a case ID. Each of the data in this case DB 44 is basically in a DICOM data format. The case server device S extracts the disease name, the date, and the like from this case DB 44 so as to generate the case browsing screen on which the plural cases are listed. The case ID is associated with each of the cases on the case browsing screen to be transmitted

In response to this, the terminal device T displays the case browsing screen on the display 55 (step S4). FIG. 6B illustrates the case browsing screen. The plural cases are displayed as a list. Although not illustrated, the cases may be searched by the disease name, an area, or the like and displayed on the list.

The operator of the terminal device T operates the keyboard/mouse 54 to select the case, browsing of which is desired, from this case list. In response to this, the terminal device T transmits the case request with the linked case ID to the case server device S (step S5). The case server device S acquires the case data from the case DB 44 on the basis of the case ID contained in this case request, and transmits the case data to the terminal device T (step S54).

The terminal device T receives this case data and displays the case data on the display 55 (step S6). In this way, as illustrated in FIG. 6C, the examination image and the like can be browsed on the terminal device T.

### 4. Case Registration Processing

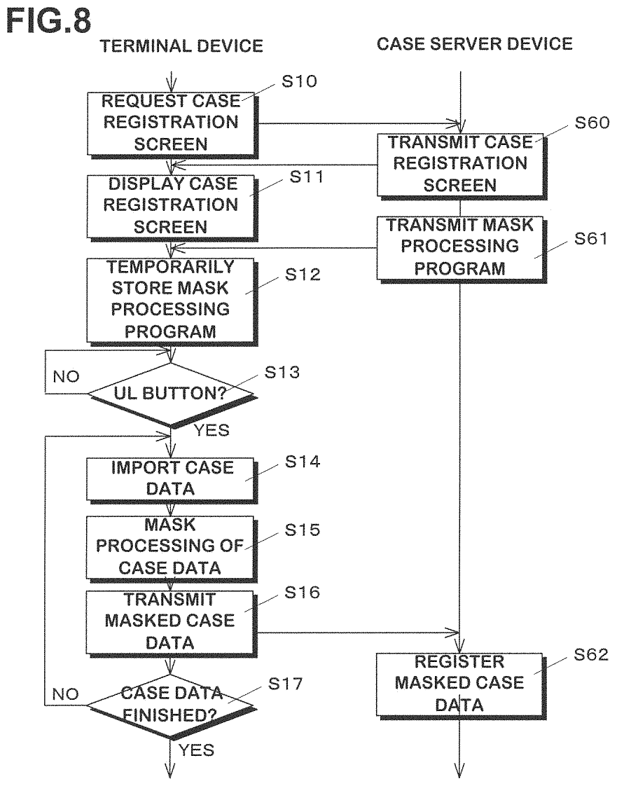

FIG. 8 is a flowchart of case data registration processing. When the case data is registered in the case DB 44 of the server device S, the case data (the DICOM data) that is a registration target recorded in a DVD-RoM 68 or the like is prepared in the terminal device T.

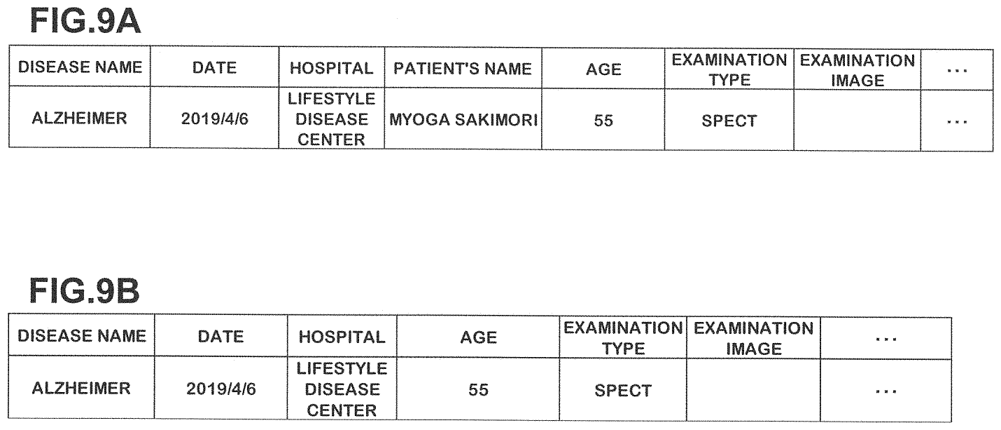

FIG. 9A illustrates an example of the case data (the DICOM data) . This DICOM data is data that is output from examination equipment such as PET or SPECT. Here, the output data from such examination equipment is recorded in the DVD-ROM 68 and is installed in the terminal device T. However, the output data may be transmitted from the examination equipment to the terminal device T via a LAN network or the like.

As illustrated in FIG. 9A, privacy items such as a patient's name are contained in the DICOM data.

Referring back to FIG. 8, it is assumed that the terminal device T is in a state where the initial screen in FIG. 6A is displayed. Here, when the operator of the terminal device T selects the case registration, the terminal device T transmits a case registration screen request to the case server device S (step S10). The case server device S receives this request and transmits a case registration screen to the terminal device T (step S60). In the terminal device T, this case registration screen is displayed on the display 55 (step S11).

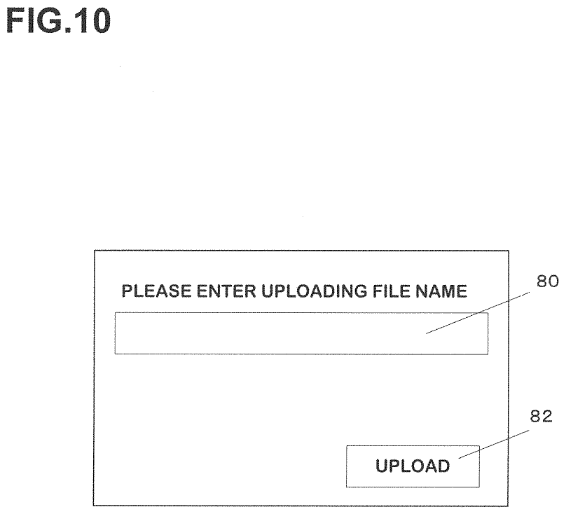

An example of the case registration screen is illustrated in FIG. 10. A file name enter area 80, in which a file name of a file to be uploaded is entered, and an upload button 82 used for uploading are provided.

The case server device S transmits the case registration screen in step S60 and also transmits the mask processing program (step S61). The flowchart in FIG. 8 is illustrated such that the case registration screen and the mask processing program are separately transmitted. However, in this embodiment, the case registration screen is transmitted in HTML (or XML), and the mask processing program is transmitted as JavaScript® contained in the HTML at the same time. Needless to say, the case registration screen and the mask processing program may be transmitted separately.

The terminal device T that has received the mask processing program temporarily stores this mask processing program in the memory 52 (step S12).

The operator of the terminal device T operates the keyboard/mouse 54 and enters the file name of the file to be uploaded in the file name enter area 80 on the case registration screen in FIG. 10. Note that the file may be selected by a pull-down menu or the like. Here, the case data that is recorded in the DVD-ROM 68 installed in the DVD-ROM drive 58 is entered as the file to be uploaded.

Next, when the operator of the terminal device T clicks the upload button 82, the terminal device T imports a part of the case data (the DICOM data in FIG. 9) as the target from the DVD-ROM 68 into the memory 52 (step S14). Due to a large volume of the case data, it is difficult to import the entire case data into the memory 52 at once. Thus, the case data is partially imported just as described.

Next, the terminal device T starts the mask processing program, which is temporarily stored in the memory 52, and executes mask processing of the privacy item in the case data (the DICOM data) by the mask processing program. In this embodiment, the mask processing is executed with the patient's name being the privacy item.

In this embodiment, while the privacy item in the DICOM data remains, the recorded data is replaced with masking characters (for example, blank characters). Accordingly, even after the mask processing, data size (file size) is not changed and thus does not cause a problem for a processing program in which the change in the data size is determined as an error.

In the above, the mask processing is executed by replacement with the masking characters. However, the mask processing may be executed by (deleting) the privacy item itself (that is, a tag) . Alternatively, the data may be deleted (for example, NULL is inserted) while the privacy item remains.

Next, the terminal device T transmits the masked case data to the case server device S (step S16).

The terminal device T repeatedly executes the processing in above steps S14 to S16 for all the items in the case data (step S17).

As it has been described so far, the masked case data (the masked DICOM data) as illustrated in FIG. 9B is transmitted from the terminal, device T to the case server device S.

The case server device 5 that has received the masked case data (the masked DICOM data) adds the case ID to the masked case data (the masked DICOM data) and adds the masked case data with the case ID (the masked DICOM data) to the case DB 44 (step S62). FIG. 7B illustrates the case DB 44 to which the new case data is added.

In this way, in regard to the case data, the case data that has undergone the mask processing is transmitted and registered even when a special program for masking is prepared on the terminal device T side.

### 5. Others

(1) In the above embodiment, the “patient's name” is the masking target. However, instead of this, or in addition to this, at least one of birth date, medical facility information, and primary doctor information may be set as the masking target. In addition, the masking target items may be determined as default in the mask processing program. Then, at the time of uploading from the terminal device T, the operator may be able to select to add (delete) the items to be masked.

(2) In the above embodiment, the mask processing program is temporarily stored. Thus, the mask processing program is deleted during transition to another processing, shut-down, or the like. That is, when the operator walks away from the case registration screen and returns to the case registration screen thereafter, the mask processing program is downloaded again.

However, in the terminal device T, the downloaded mask processing program may be recorded in the hard disk or the like. In this case, on the case registration screen, it may be determined whether the mask processing program is already downloaded and recorded. Then, in the case where the mask processing program is already recorded, the mask processing program may not be downloaded again.

(3) In the above embodiment, when the case registration screen is transmitted, the mask processing program is transmitted from the case server device at the same time.

However, the mask processing program may be transmitted when the case server device is accessed.

Alternatively, when the case server device S detects that the upload button 82 in FIG. 10 is clicked in the terminal device T, the mask processing program may be transmitted.

(4) In the above embodiment, the case DB 44 is recorded in the case server device. However, the case DB 44 may be recorded in another server device.

(5) In the above embodiment, as a mask processing method, the privacy item in which the privacy information is contained is deleted. However, a content of the privacy item may be encrypted and transmitted to the case server device S. In this case, a decryption key is recorded in the hard disk 34 on the terminal device T side by processing of the mask processing program 24. In this way, even when the case data is lost in the terminal device T, this is downloaded from the case server device S and can be recovered by the decryption key.

(6) In the above embodiment, the DICOM data is used as the case data. However, the same can be applied to the case data in another format.

(7) In the above embodiment, the system is built for a purpose of browsing the stored case data. However, a system that stores the case data can also be applied for a purpose (downloading or the like) other than browsing.

A general description of the present invention as well as preferred embodiments of the invention has been set forth above. It is to be expressly understood, however, the terms described above are for purpose of illustration only and are not intended as definitions of the limits of the invention. Those skilled in the art to which the present invention pertains will recognize and be able to practice other variations in the system, device, and methods described which fall within the teachings of this invention. Accordingly, all such modifications are deemed to be within the scope of the invention.

1-11. (canceled)

12. A case server device comprising:
upload preparation means that receives a request for an upload command related page from a terminal device and transmits said upload command related page and a mask processing program to said terminal device;
case transmission means that receives a case request from the terminal device and transmits masked case data recorded in a recording section to said terminal device; and
recording means that records the transmitted masked case data in the recording section.

13. A case server program for realizing a case server device by a computer, the case server program making the computer function as:
upload preparation means that receives a request for an upload command related page from a terminal device and transmits said upload command related page and a mask processing program to said terminal device;
case transmission means that receives a case request from the terminal device and transmits masked case data recorded in a recording section to said terminal device; and
recording means that records the transmitted masked case data in the recording section.

14. The server device according to claim 12, wherein
instead of receiving the upload command related page request, the upload preparation means acquires that the upload command is entered in the terminal device, and transmits the mask processing program.

15. The server device according to claim 12, wherein
masking is performed by deleting an item itself, deleting a content of the data, or replacing the content of the data with a masking character.

16. The server device according to claim 12, wherein
masking is performed by at least masking the item such as a patient's name, birth date, medical facility information, or primary doctor information among the items of the case data.

17. The server device according to claim 12, wherein
the case data is DICOM data.

18. A case server device comprising:
upload preparation means that receives a request for an upload command related page from a terminal device and transmits said upload command related page and a mask processing program to said terminal device; and
recording means that records the transmitted masked case data in a recording section.
